SEQUENCE LISTING (8 sequences)

SEQ ID NO: 1 - protein, 11 aa, synthetic construct
  REGION 1..11: note = Light chain variable region complementarity determining region (CDR) sequence CDR1
KASQDVSIAVA

SEQ ID NO: 2 - protein, 7 aa, synthetic construct
  REGION 1..7: note = Light chain variable region complementarity determining region (CDR) sequence CDR2
SASYRYT

SEQ ID NO: 3 - protein, 9 aa, synthetic construct
  REGION 1..9: note = Light chain variable region complementarity determining region (CDR) sequence CDR3
QQHYITPLT

SEQ ID NO: 4 - protein, 5 aa, synthetic construct
  REGION 1..5: note = Heavy chain variable region complementarity determining region (CDR) sequence CDR1
NYGMN

SEQ ID NO: 5 - protein, 17 aa, synthetic construct
  REGION 1..17: note = Heavy chain variable region complementarity determining region (CDR) sequence CDR2
WINTYTGEPTYTDDFKG

SEQ ID NO: 6 - protein, 12 aa, synthetic construct
  REGION 1..12: note = Heavy chain variable region complementarity determining region (CDR) sequence CDR3
GGFGSSYWYFDV

SEQ ID NO: 7 - protein, 214 aa, synthetic construct
  REGION 1..214: note = Light chain sequence of trop-2 targeting antibody
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 451 aa, synthetic construct
  REGION 1..451: note = Heavy chain sequence of trop-2 targeting antibody
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0263621 A1
(43) Pub. Date: Sep. 10, 2026

(54) SACITUZUMAB DRUG CONJUGATES AND PREPARATION THEREOF

(71) Applicant: ZYDUS LIFESCIENCES LIMITED, Ahmedabad, Gujarat (IN)

(72) Inventors: Sanjeev Kumar MENDIRATTA, Ahmedabad, Gujarat (IN); Sanjay BANDYOPADHYAY, Ahmedabad, Gujarat (IN); Avanish SINGH, Ahmedabad, Gujarat (IN); Pankaj KALITA, Ahmedabad, Gujarat (IN); Swagat SONI, Ahmedabad, Gujarat (IN)

(21) Appl. No.: 19/166,400

(22) PCT Filed: Mar. 16, 2024
(86) PCT No.: PCT/IB2024/052569

§ 371 Date: Sep. 17, 2025

(30) Foreign Application Priority Data
Mar. 17, 2023 (IN) 202321018198

(51) Int. Cl. A61K 47/68

(52) U.S. Cl. CPC A61K 47/6851; A61K 47/68031; A61K 47/6889

(57) ABSTRACT

The invention provides antibody-linker-drug conjugates wherein antibody is sacituzumab and drug is auristatin analogues or a pharmaceutically acceptable salt thereof. The invention also provides a process of preparing antibody-linker-conjugates of the present invention.

### CROSS-REFERENCE TO RELATED APPLICATIONS

This application is the U.S. national phase of International Application No. PCT/IB2024/052569 filed Mar. 16, 2024 which designated the U.S. and claims priority to Indian Application No. 202321018198 filed Mar. 17, 2023, the entire contents of each of which are hereby incorporated by reference.

### REFERENCE TO SEQUENCE LISTING SUBMITTED ELECTRONICALLY

The content of the electronically submitted sequence listing (Name: 9001-121_Sequence Listing.xml, Size: 12,066 bytes, and Date of Creation: Sep. 16, 2025) is herein incorporated by reference in its entirety.

### FIELD OF THE INVENTION

The invention provides antibody-linker-drug conjugate wherein antibody is targeted to Trop-2 antigen, preferably, the antibody is Sacituzumab and drug is auristatin analogues.

### BACKGROUND OF THE INVENTION

Antibody drug conjugates (ADC's), composed of an antibody, linker, and cytotoxic agent (drug), are one of the most complex drug platforms in the oncology armamentarium^{[1]}. They are a payload delivery system with major variables influencing their success including: (i) the rate of internalization of the payload; (ii) the expression of the target antigen on the tumor and normal tissues with the implications for both patient selection as well as therapeutic index; (iii) the linker chemistry and, implicit within the choice of chemistry, the extracellular as well as intracellular stability; and (iv) the selection of payload for the tumor indication^{[2]}. The mechanisms of action of ADCs include direct binding between the antibody and its targeted cell surface antigen, and intracellular or extracellular release of cytotoxic drugs^{[3]}. Present invention provides sacituzumab derived antibody drug conjugates to enable specific delivery of drug (auristatin and/or its analogues) to target antigen on cancer cells.

### SUMMARY OF THE INVENTION

The present invention provides antibody-linker-drug conjugates wherein antibody is trop-2 antigen targeting antibody, preferably sacituzumab and the drug is suitable auristatin analogues. The present invention provides higher efficacy than approved therapy in the area of trop-2 expressing cancers. The invention also provides a process of preparing antibody-linker-drug conjugates of the present invention. The present invention is also directed to antibody-linker-drug conjugates in the treatment of certain types of cancer, an autoimmune disease or an infectious disease.

### Abbreviations

Ab: Antibody
ADC: Antibody drug conjugate or Antibody-linker-drug conjugate
HP-SEC: High performance size-exclusion chromatography
HP-HIC: High-performance hydrophobic interaction chromatography
HMW: High molecular weight
IRS: Internal reference standard
LMW: Low molecular weight
MMAE: Monomethyl auristatin E
MMAF: Monomethyl auristatin F
mc-vc-PABC: maleimido caproyl valine citrulline para amino benzyl carbamate
PAB: Para amino benzyl
PABC: Para amino benzyl carbamate
SDS PAGE: Sodium dodecyl-sulfate polyacrylamide gel electrophoresis
TCEP: Tris (2-carboxyethyl) phosphine

### EMBODIMENTS OF THE INVENTION

### Embodiment 1

In one embodiment, the present invention provides antibody-linker-drug conjugates (ADC) of Formula (I): Ab [symbol] Aa-Ww-Yy-D)ₚ wherein antibody (Ab) is a trop-2 targeting antibody, preferably, sacituzumab; -Aa-Ww-Yy- is an enzymatically cleavable linker unit that links the drug unit and the antibody, wherein: -A- is a stretcher unit; a is 1; each -W- is independently an amino acid unit; -Y- is a spacer unit; w is an integer ranging from 2 to 12, y is 1 or 2; p ranges from 1 to about 20; and drug (D) is a suitable auristatin analogue selected from MMAE and MMAF as defined by following structures;

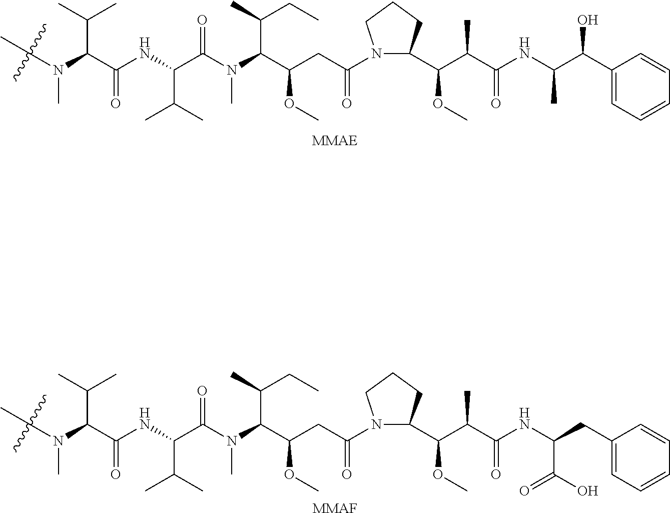

Wherein wavy line represent connection point to the linker.

In a further embodiment, the present invention provides antibody-linker-drug conjugates of formula (I) Ab [symbol] Aa-Ww-Yy-D)ₚ wherein antibody (Ab) is sacituzumab; -Aa-Ww-Yy- is an enzymatically cleavable linker unit that links the drug unit and the antibody, wherein: -A- is a stretcher unit; a is 1; each -W- is independently an amino acid unit selected from -phenylalanine-lysine- or -valine-citrulline-; -Y- is a spacer unit selected from -glycine-glycine- or p-amino benzyl alcohol (PAB) or p-aminobenzyl carbamate (PABC); w is an integer ranging from 2 to 12, y is 1 or 2; p ranges from 1 to about 20; and drug (D) is selected from MMAE and MMAF as defined by following structures:

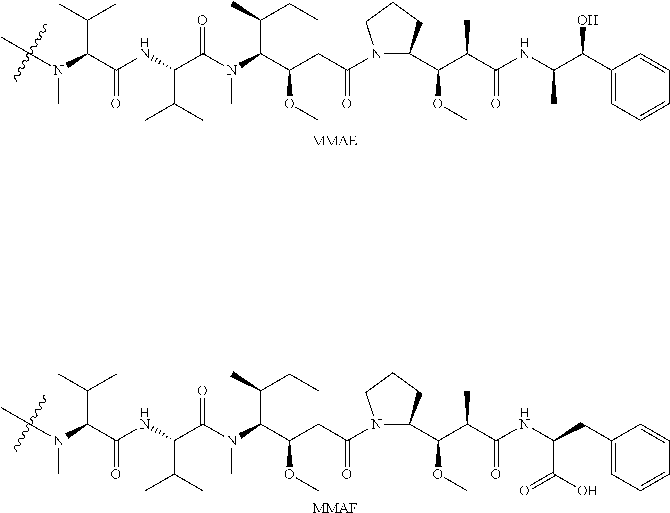

Wherein wavy line represents connection point to the linker,
Wherein A is defined by following structure;

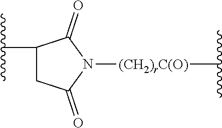

r is an integer ranging from 1 to 10; wherein carbonyl terminus of -A- forming bond with an amino acid unit (W) and the succinimido terminus of -A- forming bond with antibody (Ab).

In preferred embodiment, the present invention provides an antibody drug conjugate (ADC) structure represented by;

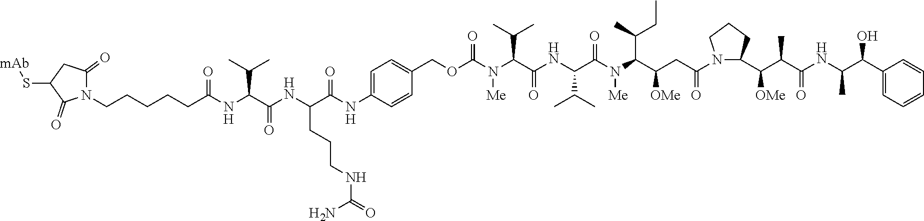

wherein mAb is a monoclonal antibody comprising light chain variable region complementarity determining region (CDR) sequences CDR1 (KASQDVSIAVA, SEQ ID NO: 1), CDR2 (SASYRYT, SEQ ID NO: 2) and CDR3 (QQHYITPLT, SEQ ID NO: 3) and heavy chain variable region CDR sequences CDR1 (NYGMN, SEQ ID NO: 4), CDR2 (WINTYTGEPTYTDDFKG, SEQ ID NO: 5) and CDR3 (GGFGSSYWYFDV, SEQ ID NO: 6). In this embodiment, the antibody is Sacituzumab.

In preferred embodiment, the present invention provides, an antibody-linker-drug conjugate (ADC) wherein antibody is a monoclonal antibody comprising light chain variable region complementarity determining region (CDR) sequences CDR1 (KASQDVSIAVA, SEQ ID NO: 1), CDR2 (SASYRYT, SEQ ID NO: 2) and CDR3 (QQHYITPLT, SEQ ID NO: 3) and heavy chain variable region CDR sequences CDR1 (NYGMN, SEQ ID NO: 4), CDR2 (WINTYTGEPTYTDDFKG, SEQ ID NO: 5) and CDR3 (GGFGSSYWYFDV, SEQ ID NO: 6); linker is enzymatically cleavable linker unit mc-vc-PABC and drug (D) is a suitable auristatin analogue MMAE; wherein said monoclonal antibody (mAb) is attached to between 4.2 and 4.4 drug moieties.

In preferred embodiment, the present invention provides, the antibody-linker-drug conjugates (ADC), wherein the ADC structure is represented by;

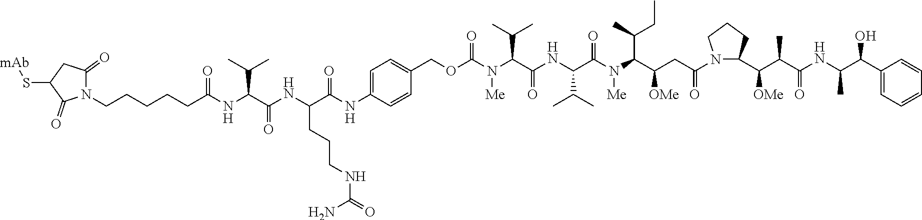

Wherein mAb is a monoclonal antibody comprising light chain variable region complementarity determining region (CDR) sequences CDR1 (KASQDVSIAVA, SEQ ID NO: 1), CDR2 (SASYRYT, SEQ ID NO: 2) and CDR3 (QQHYITPLT, SEQ ID NO: 3) and heavy chain variable region CDR sequences CDR1 (NYGMN, SEQ ID NO: 4), CDR2 (WINTYTGEPTYTDDFKG, SEQ ID NO: 5) and CDR3 (GGFGSSYWYFDV, SEQ ID NO: 6) wherein said monoclonal antibody (mAb) is attached to between 4.2 and 4.4 drug moieties.

In preferred embodiment, the present invention provides, the antibody drug conjugate (ADC) wherein said antibody (mAb) is attached to 4.2 drug moieties (i.e. DAR 4.2), about 4.3 drug moieties (i.e. DAR about 4.3) or 4.4 drug moieties (i.e. DAR 4.4).

### Further Embodiments

In a second embodiment, the present invention provides a pharmaceutical composition comprising antibody drug conjugate, preferably sacituzumab drug conjugate as embodied in embodiment 1 and an acceptable carrier. Acceptable carrier is defined as any suitable pharmaceutical excipient known in the art^{5,6} and which is compatible with active pharmaceutical ingredient (API), i.e. antibody drug conjugate, herein of the present invention.

In a third embodiment, the present invention provides a process for preparing the antibody drug conjugate, preferably sacituzumab drug conjugate as embodied in embodiment 1 wherein process comprising a) purification of antibody Sacituzumab, b) partial reduction of sacituzumab c) conjugation of the partially reduced sacituzumab with vc-MMAE drug linker.

In a further embodiment, the conjugate according to the present invention, wherein the monoclonal antibody is reactive with an antigen or epitope of an antigen, preferably trop-2, associated with a cancer, a malignant cell, an autoimmune disease or an infectious organism expressing a Trop-2 cross-reactive epitope for binding to the antibody-linker-drug conjugates of this invention.

In a further embodiment, the sacituzumab drug conjugate according to the present invention may be used in the treatment of cancer.

In a still further embodiment, the sacituzumab drug conjugate according to the present invention may be used in the treatment of an autoimmune disease or an infectious disease.

In a further embodiment, the sacituzumab drug conjugate according to the present invention may be used alone as a monotherapy in the treatment of cancer.

In a still further embodiment, the sacituzumab drug conjugate according to the present invention may be used alone as a monotherapy in the treatment of an autoimmune disease or an infectious disease.

In a further embodiment, the sacituzumab drug conjugate according to the present invention may be used in combination with other drug products in the treatment of cancer, an autoimmune disease or an infectious disease.

In further embodiment, the conjugate of the present invention may be administered either as an intravenous (i.v.), intramuscular (i.m.), subcutaneous (s.c.), intraperitoneal (i.p.) or any other suitable route of administration.

In a further embodiment, the dose of the antibody drug conjugate (ADC) according to the present invention may be in the range of 0.1 mg/kg to 10 mg/kg.

### Definitions

The term “linker” is any chemical moiety that is capable of linking a compound, usually a drug, such as auristatin, to a cell-binding agent such as sacituzumab in covalent manner. The linker includes a stretcher unit (-A-), an amino acid unit (-W-), and a spacer unit (-Y-) as defined by formula;

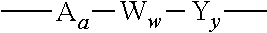

Linkers can be susceptible to or be substantially resistant to acid-induced cleavage, light-induced cleavage, peptidase-induced cleavage, esterase-induced cleavage, and disulfide bond cleavage, at conditions under which the compound or the antibody remains active. Suitable linkers are well known in the art and include, for example, disulfide groups, thioether groups, acid labile groups, photolabile groups, peptidase labile groups and esterase labile groups. Linkers also include charged linkers, and hydrophilic forms thereof as described herein and known in the art.

The term “pharmaceutical composition” refers to preparations which are in such form as to permit the biological activity of the active ingredients to be unequivocally effective, and which contain no additional components which are significantly toxic to the subjects to which the formulation would be administered. The term “pharmaceutical formulation”, “formulation”, “pharmaceutical composition”, or “composition” can be used here interchangeably.

The terms “patient” and “subject” are used interchangeably and are used in their conventional sense to refer to a living organism suffering from or prone to a condition that can be prevented or treated by administration of a composition of the present invention, and includes animals. The term “Animal” refers to a human or non-human animal, including, but not limited to, farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese and non-human primates, including, but not limited to, monkeys, chimpanzees and other apes and monkey species. This term does not denote a particular age. Thus, adult, juvenile and newborn individuals are of interest.

In the context of cancer, the term “treating” includes any or all of: killing of tumor cells or cancer cells, preventing growth of tumor cells or cancer cells, regression of tumor cells or cancer cells, preventing replication of tumor cells or cancer cells, lessening of overall tumor burden and ameliorating one or more symptoms associated with the disease.

In the context of an autoimmune disease, the term “treating” includes any or all of: killing of cells producing antibody directly or those cells that help antibody producing cells in their action, preventing replication of cells associated with an autoimmune disease state including, but not limited to, cells capable of producing an autoimmune antibody, lessening the autoimmune-antibody burden and ameliorating one or more symptoms of an autoimmune disease.

In the context of an infectious disease, the term “treating” includes any or all of: preventing the growth, multiplication or replication of the pathogen that causes the infectious disease and/or pathogen-infected host cells and ameliorating one or more symptoms of an infectious disease.

The phrase “pharmaceutically acceptable salt,” as used herein, refers to pharmaceutically acceptable organic or inorganic salts of a drug (payload) of the present invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate salts.

Unless otherwise specified, a or an means “one or more.”

An antibody drug conjugate (ADC) is defined as an antibody component or fragment thereof conjugated to a therapeutic agent. As used herein, the terms “antibody drug conjugate”, “antibody-linker-drug” “conjugate” and “immunoconjugate” are used interchangeably herein the present invention. The preferred antibody drug conjugate of the present invention is sacituzumab-MMAE conjugate. The “sacituzumab-MMAE conjugate” and “sacituzumab vedotin” are used interchangeably.

As used herein, the term “about” when used in reference to a specifically recited value means that the value may vary by ±0.1 from the recited value. For example, the expression “about 4.3” includes 4.2 to 4.4 and all integers and non-integers values in between. The specific values within the range between 4.2 and 4.4 includes, but not limited to 4.20, 4.21, 4.22, 4.23, 4.24, 4.25, 4.26, 4.27, 4.28, 4.29, 4.30, 4.31, 4.32, 4.33, 4.34, 4.35, 4.36, 4.37, 4.38, 4.39 or 4.40.

### DESCRIPTION OF ACCOMPANYING FIGURES

FIG. 1: FIG. 1 demonstrate purity and size variants profile of Sacituzumab-MMAE conjugate evaluated by HP-SEC

FIG. 2: FIG. 2 demonstrate drug antibody ratio (DAR) of Sacituzumab-MMAE conjugates analysed by HP-HIC

FIG. 3: FIG. 3 demonstrates the polypeptide profile of sacituzumab-MMAE conjugate of the present invention by SDS-PAGE analysis.

FIG. 4: FIG. 4 demonstrates in-vitro cytotoxicity assay of sacituzumab-MMAE conjugate using BxPC3 cells.

FIG. 5: FIG. 5 demonstrates in-vitro cytotoxicity assay of sacituzumab-MMAE conjugate using NCI-N87 cells.

FIG. 6: FIG. 6 demonstrates in-vitro cytotoxicity assay of sacituzumab-MMAE conjugate using HCC1806 cells.

FIG. 7: FIG. 7 demonstrates in-vitro cytotoxicity assay of sacituzumab-MMAE conjugate using MDA-MB-468 cells.

FIG. 8: FIG. 8 demonstrates In-vivo efficacy of Sacituzumab-MMAE conjugate in gastric carcinoma xenograft disease model in SCID mice

FIG. 9: FIG. 9 demonstrates In-vivo efficacy of Sacituzumab-MMAE conjugate in BRCA mutated triple negative breast cancer xenograft disease model in SCID mice

### DETAILED DESCRIPTION OF THE PRESENT INVENTION

The present invention provides antibody-linker-drug conjugates of Ab [symbol] Aa-Ww-Yy-D)ₚ wherein antibody (Ab) is trop-2 antigen targeting antibody, preferably sacituzumab; -Aa-Ww-Yy- is an enzymatically cleavable linker unit that links the drug unit and the antibody, wherein: -A- is a stretcher unit; a is 1; each -W- is independently an amino acid unit; -Y- is a spacer unit; w is an integer ranging from 2 to 12, y is 1 or 2; p ranges from 1 to about 20; and drug (D) is auristatin analogues or a pharmaceutically acceptable salt thereof.

### Antibody (Ab):

The antibody of the antibody drug conjugates (ADC) of the present invention is trop-2 targeting antibody, preferably sacituzumab or any posttranslational modification of an antibody or antibody variant thereof. The posttranslational modification of the antibody includes, but not limited to, deamidated, acidic, basic or oxidized variants or any other variants of an antibody that do not impact the biological function or efficacy of the antibody. In one embodiment, the antibody of the antibody drug conjugates (ADC) of the present invention is sacituzumab variants. Sacituzumab variants of the present invention include an amino acid substitution, insertion, and/or deletion in a polypeptide sequence of the antibody sacituzumab. In one embodiment, the trop-2 targeting antibody according to the present invention is an antibody that comprises light chain variable region complementarity determining region (CDR) sequences CDR1 (KASQDVSIAVA, SEQ ID NO: 1), CDR2 (SASYRYT, SEQ ID NO: 2) and CDR3 (QQHYITPLT, SEQ ID NO: 3) and heavy chain variable region CDR sequences CDR1 (NYGMN, SEQ ID NO: 4), CDR2 (WINTYTGEPTYTDDFKG, SEQ ID NO: 5) and CDR3 (GGFGSSYWYFDV, SEQ ID NO: 6). In preferred embodiment, the trop-2 targeting antibody according to the present invention is an antibody that comprises light chain sequence SEQ ID NO: 7 (DIQLTQSPSS LSASVGDRVS ITCKASQDVS IAVAWYQQKP GKAPKLLIYS ASYRYTGVPD RFSGSGSGTD FTLTISSLOP EDFAVYYCQQ HYITPLTFGA GTKVEIKRTV AAPSVFIFPP SDEQLKSGTA SVVCLLNNFY PREAKVQWKV DNALQSGNSQ ESVTEQDSKD STYSLSSTLT LSKADYEKHK VYACEVTHQG LSSPVTKSEN RGEC) and heavy chain SEQ ID NO: 8 (QVQLQQSGSE LKKPGASVKV SCKASGYTFT NYGMNWVKQA PGQGLKWMGW INTYTGEPTY TDDFKGRFAF SLDTSVSTAY LQISSLKADD TAVYFCARGG FGSSYWYFDV WGQGSLVTVS SASTKGPSVF PLAPSSKSTS GGTAALGCLV KDYFPEPVTV SWNSGALTSG VHTFPAVLQS SGLYSLSSVV TVPSSSLGTQ TYICNVNHKP SNTKVDKRVE PKSCDKTHTC PPCPAPELLG GPSVFLFPPK PKDTLMISRT PEVTCVVVDV SHEDPEVKEN WYVDGVEVHN AKTKPREEQY NSTYRVVSVL TVLHQDWLNG KEYKCKVSNK ALPAPIEKTI SKAKGQPREP QVYTLPPSRE EMTKNQVSLT CLVKGFYPSD IAVEWESNGQ PENNYKTTPP VLDSDGSFFL YSKLTVDKSR WQQGNVFSCS VMHEALHNHY TQKSLSLSPG K) that represent sacituzumab antibody. The said sequences have been disclosed in International Nonproprietary Names for Pharmaceutical Substances (INN, Vol. 29, No. 2, 2015^{[4]} for sacituzumab product. The sacituzumab is an immunoglobulin G1 kappa (IgG1κ) humanized monoclonal antibody against Trop-2 antigen, produced in a mammalian cell line (Chinese Hamster Ovary [CHO]) using recombinant DNA technology. In one embodiment, the antibody of the antibody drug conjugates (ADC) of the present invention is sacituzumab or its variants that can improve binding affinity of ADC to the trop-2 antigen. In one more embodiment, the antibody of the antibody drug conjugates (ADC) of the present invention is sacituzumab or its variants that can improve binding affinity of ADC to the trop-2 antigen at suitable pH. The preferred pH can be pH 6.0 or pH 7.0. In another embodiment, the antibody of the present invention is sacituzumab or its variants that can be used to increase the half-life of ADC. The half-life of an ADC of the present invention can be modified in blood by mutation in the Fc domain of the antibody. The mutation in Fc domain includes but not limited to one or more amino acid substitution, insertion, and/or deletion in amino acid sequence of the antibody.

### The Linker Unit:

The Linker unit of the Antibody-Linker-Drug Conjugate links the Drug unit and the antibody unit and has the formula:

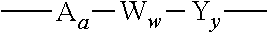

Wherein:
   -A- is a stretcher unit that linked with antibody;
   a is 0 or 1;
   each -W- is independently an amino acid unit;
   w is independently an integer ranging from 0 to 12;
   -Y- is a spacer unit that linked with drug/payload; and
   Y is 0, 1 or 2.

The stretcher unit: The Stretcher unit (-A-), when present, links an antibody unit to an amino acid unit (-W-). In this regard antibody (Ab) has a functional group that can form a bond with a functional group of a Stretcher. Useful functional groups that can be present on an antibody, either naturally or via chemical manipulation include, but are not limited to, sulfhydryl (—SH), amino, hydroxyl, carboxy, the anomeric hydroxyl group of a carbohydrate, and carboxyl. Preferred antibody functional groups are sulfhydryl and amino.

The stretcher unit (-A-) of the present invention is defined by following structure;

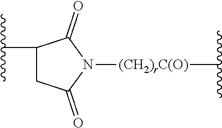

r is an integer ranging from 1 to 10; wherein carbonyl terminus of -A- forming bond with an amino acid unit (W) and the succinimido terminus of -A- forming bond with antibody (Ab).

In one embodiment, the Stretcher unit forms a bond with a sulfur atom of the antibody unit. The sulfur atom can be derived from a sulfhydryl group of antibody. Representative Stretcher units of this embodiment are depicted as below;

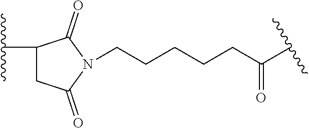 [SMILES: *C(=O)CCCCCN1C(=O)CC(*)C1=O]

The amino acid unit (-W-): The Amino Acid unit (-W-), when present, links the Stretcher unit to the Spacer unit if the Spacer unit is present, links the Stretcher unit to the Drug unit if the Spacer unit is absent, and links the antibody unit to the Drug unit if the Stretcher unit and Spacer unit are absent. Wavy line represent point of connection.

-Ww- is a dipeptide, tripeptide, tetrapeptide, pentapeptide, hexapeptide, heptapeptide, octapeptide, nonapeptide, decapeptide, undecapeptide or dodecapeptide unit. The Amino Acid unit of the ADC of the invention can be enzymatically cleaved by one or more enzymes, including a tumor-associated protease, to liberate the Drug unit (-D). Illustrative Ww units are represented by formula;

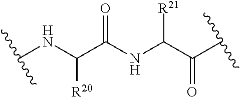

Wherein R²⁰ is isopropyl and R²¹ is (CH₂)₃NHCONH₂.

The Spacer unit: The Spacer unit (-Y-), when present, links an amino acid unit to the drug unit when an amino acid unit is present. Alternately, the Spacer unit links the Stretcher unit to the Drug unit when the amino acid unit is absent. The Spacer unit also links the Drug unit to the antibody unit when both the amino acid unit and stretcher unit are absent. Spacer units are of two general types: self-immolative and non-self-immolative. In one embodiment, the Spacer unit (-Y-) of the present invention can be self-immolative or non-self-immolative. A non-self-immolative Spacer unit is one in which part or all of the Spacer unit remains bound to the Drug unit after cleavage, particularly enzymatic cleavage of an amino acid unit from the antibody-linker-drug conjugate. Examples of a non-self-immolative Spacer unit include, but are not limited to a (glycine-glycine) Spacer unit and a glycine Spacer unit. When a Compound of the invention containing a glycine-glycine Spacer unit or a glycine Spacer unit undergoes enzymatic cleavage via a tumor-cell associated-protease, a cancer-cell-associated protease or a lymphocyte-associated protease, a glycine-glycine-Drug moiety or a glycine-Drug moiety is cleaved from Ab-Aa-Ww-. In one embodiment, -Y_{y}- is a p-aminobenzyl alcohol (PAB) unit. In one embodiment, the invention provides a Drug-Linker Compound or antibody-Linker drug conjugate in which the Spacer unit is absent (=0). In one embodiment, -Y- is a p-aminobenzyl alcohol (PAB) group that is linked to -W_{w}- via the amino nitrogen atom of the PAB group, and connected directly to -D via a carbonate, carbamate or ether group.

The drug unit: The present invention provides antibody drug conjugate wherein antibody is sacituzumab and drug is auristatin analogues selected from MMAE and MMAF as defined by following formulas:

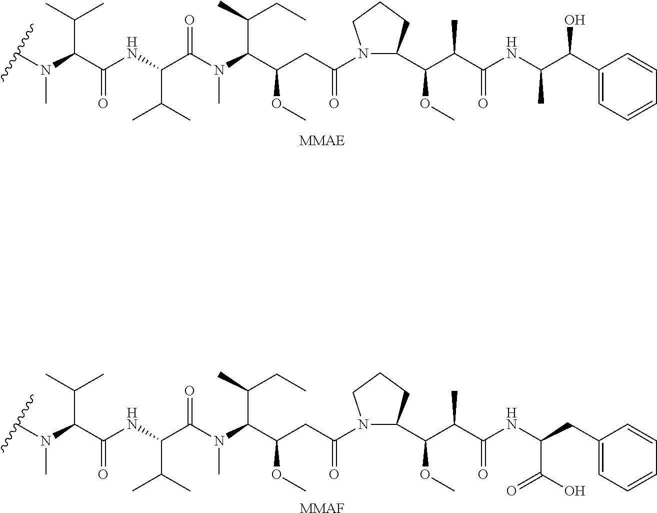

Wherein wavy line represent connection point to the linker.

### Antibody Drug Conjugate of the Present Invention

The present invention provides antibody drug conjugate wherein the antibody is sacituzumab or its variant that targets trop-2 antigen and drug or payload is an auristatin analogue preferably MMAE. The preferred antibody drug conjugate of the present invention is sacituzumab-MMAE conjugate also named as sacituzumab vedotin. The sacituzumab-MMAE conjugate of the present invention comprises sacituzumab or its variants as an antibody, mc-vc-PABC as a linker and MMAE as a toxin or drug or payload. The conjugate of the present invention also includes the pharmaceutical salt of the payload i.e. MMAE. The inventors of the present invention have found that the sacituzumab-MMAE conjugates of the present invention produce significantly better biological activity in in-vitro as well as in-vivo studies in xenograft animal models as compared to an approved drug conjugate therapy (Trodelvy®) in trop-2 expressing cancer. It is expected that the biological activity of sacituzumab-MMAE of the present invention will be extrapolated to clinical use in humans. The novel drug conjugate of the present invention is better than the approved drug conjugate, Trodelvy®, in that it can treat the BRCA mutation bearing tumors by alone itself (Example 8) without the need for a second co-treatment e.g. PARP inhibitors to be given along with the primary treatment as in Trodelvy®⁷. The novel drug conjugate of the present invention is also better than the approved drug conjugate in that it is efficacious at significantly lower drug concentrations making the drug cheaper for the patients and possibly less toxic while being equally or better efficacious than the approved drug conjugate i.e. Trodelvy®.

### Process of Preparing Antibody Drug Conjugate of the Present Invention:

Antibody of the present invention, preferably sacituzumab was expressed in CHO (Chinese hamster ovary) cells using recombinant technology. Cells were cultured in cell culture media at suitable pH and temperature for specific period. After completion of cell incubation, cell cultures were harvested and antibody was isolated and purified by suitable chromatography technique. The antibody, preferably sacituzumab, of the present invention was reduced with reducing agents such as Tris (2-carboxyethyl)phosphine (TCEP) in phosphate buffer at pH in the range of 5-8.5 at temperature about 37° C. for 120 minutes. Alternatively, sacituzumab of the present invention can be reduced by dithiothreitol (DTT). The reduced sacituzumab was reacted with about 5 to 10 molar excess of linker drug conjugate of the present invention. Linker drug conjugate of the present invention is vedotin, which is commercially available in the market. Vedotin of the present invention comprises mc-vc-PABC linker conjugated with monomethyl auristatin (MMAE) payload, also known as vc-MMAE or mc-vc-PABC-MMAE. The conjugate was purified by ultrafiltration-diafiltration. The drug antibody ratios (DAR) were determined by HPLC-HIC column method. The drug antibody ratios (DAR) obtained for sacituzumab vedotin of the present invention, which were typically in the 1 to 8 range DAR for sacituzumab vedotin of the present invention in the 3 to 5 range or in the range of 3.5 to 4.5.

In an embodiment, the present invention provides antibody drug conjugate (ADC), wherein said antibody (mAb) is attached to between 1 and 8 drug moieties, preferably between 4.2 and 4.4 drug moiety. The specific values within the range between 4.2 and 4.4 includes but not limited to, 4.20, 4.21, 4.22, 4.23, 4.24, 4.25, 4.26, 4.27, 4.28, 4.29, 4.30, 4.31, 4.32, 4.33, 4.34, 4.35, 4.36, 4.37, 4.38, 4.39 or 4.40.

In preferred embodiment, the present invention provides, an antibody-linker-drug conjugates (ADC) wherein antibody is a monoclonal antibody comprising light chain variable region complementarity determining region (CDR) sequences CDR1 (KASQDVSIAVA, SEQ ID NO: 1), CDR2 (SASYRYT, SEQ ID NO: 2) and CDR3 (QQHYITPLT, SEQ ID NO: 3) and heavy chain variable region CDR sequences CDR1 (NYGMN, SEQ ID NO: 4), CDR2 (WINTYTGEPTYTDDFKG, SEQ ID NO: 5) and CDR3 (GGFGSSYWYFDV, SEQ ID NO: 6); linker is enzymatically cleavable linker unit mc-vc-PABC and drug (D) is a suitable auristatin analogue MMAE; wherein said monoclonal antibody (mAb) is attached to between 4.2 and 4.4 drug moieties. In preferred embodiment, the present invention provides, the antibody-linker-drug conjugates (ADC), wherein the ADC structure represented by;

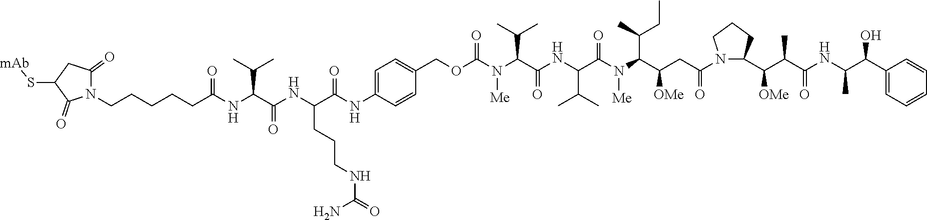

Wherein mAb is a monoclonal antibody comprising light chain variable region complementarity determining region (CDR) sequences CDR1 (KASQDVSIAVA, SEQ ID NO: 1), CDR2 (SASYRYT, SEQ ID NO: 2) and CDR3 (QQHYITPLT, SEQ ID NO: 3) and heavy chain variable region CDR sequences CDR1 (NYGMN, SEQ ID NO: 4), CDR2 (WINTYTGEPTYTDDFKG, SEQ ID NO: 5) and CDR3 (GGFGSSYWYFDV, SEQ ID NO: 6) wherein said monoclonal antibody (mAb) is attached to between 4.2 and 4.4 drug moieties.

In preferred embodiment, the present invention provides, the antibody drug conjugate (ADC) wherein said antibody (mAb) is attached to 4.2 drug moieties, about 4.3 drug moieties or 4.4 drug moieties. In preferred embodiment, the drug antibody ratio (DAR) of the ADC of the present invention is 4.20, 4.21, 4.22, 4.23, 4.24, 4.25, 4.26, 4.27, 4.28, 4.29, 4.30, 4.31, 4.32, 4.33, 4.34, 4.35, 4.36, 4.37, 4.38, 4.39 or 4.40.

In one embodiment, the present invention provides, the antibody drug conjugate (ADC) wherein said antibody (mAb) is attached to 4.20, 4.21, 4.22, 4.23, 4.24, 4.25, 4.26, 4.27, 4.28, 4.29, 4.30, 4.31, 4.32, 4.33, 4.34, 4.35, 4.36, 4.37, 4.38, 4.39 or 4.40 drug moieties.

For long-term storage, the purified drug conjugate aliquots have been stored under frozen condition, at −25±5° C.

### Use of the Conjugate of the Present Invention:

In another aspect, the antibody drug conjugate of the present invention may be used in the treatment of a subject, comprising administering a therapeutically effective amount of a therapeutic conjugate, preferably sacituzumab drug conjugate, as described herein to a subject. The effective dose of the conjugate may be in the range of 0.1 mg/kg to 10 mg/kg. Diseases that may be treated with the sacituzumab drug conjugate include, but are not limited to, cancers, autoimmune diseases and/or infectious diseases. In one aspect, the conjugate of the present invention may be used in the treatment of pancreatic cancer, breast cancer and/or gastric cancer. In one aspect, the conjugate of the present invention may be used in the treatment of BRCA mutated triple negative breast cancer, carcinoma, lymphoma, glioblastoma, melanoma, sarcoma, and leukemia, myeloma, or lymphoid malignancies. More particular examples of such cancers are noted below and include: squamous cell cancer (e.g., epithelial squamous cell cancer), Ewing sarcoma, Wilms tumor, astrocytomas, glioblastoma multiforme, cervical cancer, Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Adult Acute Lymphocytic Leukemia, Adult Acute Myeloid Leukemia, Adult Hodgkin's Lymphoma, Adult Lymphocytic Leukemia, Adult Non-Hodgkin's Lymphoma, AIDS-Related Lymphoma, AIDS-Related Malignancies, Central Nervous System (Primary) Lymphoma, Central Nervous System Lymphoma, Cerebellar Astrocytoma, Cerebral Astrocytoma, Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Cutaneous T-Cell Lymphoma, Endocrine Pancreas Islet Cell Carcinoma, Endometrial Cancer, Ependymoma, Epithelial Cancer, Ewing's Sarcoma and Related Tumors, Extracranial Germ Cell Tumor, Extragonadal Germ Cell Tumor, Eye Cancer, Gaucher's Disease, Hodgkin's Lymphoma, Hypergammaglobulinemia, Hypopharyngeal Cancer, Intraocular Melanoma, Kaposi's Sarcoma, Lymphoproliferative Disorders, Macroglobulinemia, Malignant Mesothelioma, Malignant Thymoma, Medulloblastoma, Melanoma, Mesothelioma, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma, Nonmelanoma Skin Cancer, Osteo-/Malignant Fibrous Sarcoma, Osteosarcoma/Malignant Fibrous Histiocytoma, Osteosarcoma/Malignant Fibrous Histiocytoma of Bone, Ovarian Germ Cell Tumor, Ovarian Low Malignant Potential Tumor, Paraproteinemias, Polycythemia vera, Parathyroid Cancer, Penile Cancer, Pheochromocytoma, Pituitary Tumor, Primary Central Nervous System Lymphoma, Retinoblastoma, Rhabdomyosarcoma, Salivary Gland Cancer, Sarcoidosis Sarcomas, Sezary Syndrome, Skin Cancer, Testicular Cancer, Thymoma, Thyroid Cancer, Waldenstrom's Macroglobulinemia, Wilms' Tumor, and any other hyper proliferative disease, besides neoplasia, located in an organ system listed above. The ADC of the present invention can be given once or repeatedly, depending on the disease state and tolerability of the conjugate, and can be used alone as a monotherapy or in combination with other therapeutic modalities, such as surgery, external radiation, radio immunotherapy, immunotherapy, chemotherapy, antisense therapy, interference RNA therapy, gene therapy, and the like. Each combination will be adapted to the tumor type, stage, patient condition and prior therapy, and other factors considered by the managing physician.

### Formulation and Administration

Suitable routes of administration of the sacituzumab drug conjugates of the present invention include, but not limited to, oral, parenteral, rectal, transmucosal, intestinal administration, intramuscular, subcutaneous, intramedullary, intrathecal, direct intraventricular, intravenous, intravitreal, intraperitoneal, intranasal, or intraocular injections. The preferred routes of administration are parenteral. Alternatively, one may administer the compound in a local rather than systemic manner, for example, via injection of the compound directly into a solid tumor.

The antibody drug conjugate, preferably sacituzumab drug conjugate of the present invention can be formulated according to known methods to prepare pharmaceutically useful compositions, whereby the antibody drug conjugate is combined in a mixture with a pharmaceutically suitable excipient. Sterile phosphate-buffered saline is one example of a pharmaceutically suitable excipient. Other suitable excipients are well known to those in the art.^{5, 6}

In an embodiment, the present invention provides antibody-linker-drug conjugates (ADC) of Formula (I):

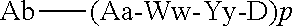

wherein antibody (Ab) is a trop-2 targeting antibody;
-Aa-Ww-Yy- is an enzymatically cleavable linker unit that links the drug unit (D) and the antibody, wherein: -A- is a stretcher unit; a is 1; each -W- is independently an amino acid unit; -Y- is a spacer unit; w is an integer ranging from 2 to 12, y is 1 or 2; p ranges from 1 to about 20; and drug (D) is a suitable auristatin analogue selected from MMAE and MMAF as defined by following structures;

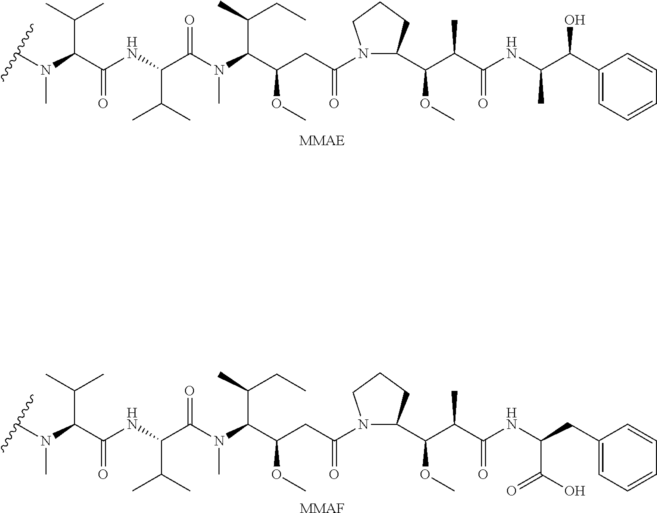

wherein wavy line represent connection point to the linker.

In another embodiment, the present invention provides antibody-linker-drug conjugates wherein: -A- is a stretcher unit; -W- is independently an amino acid unit selected from -phenylalanine-lysine- or -valine-citrulline-; -Y- is a spacer unit selected from -glycine-glycine- or p-amino benzyl alcohol (PAB) or p-aminobenzyl carbamate (PABC).

In another embodiment, the present invention provides antibody-linker-drug conjugates wherein: -A- is a stretcher unit defined by following structure;

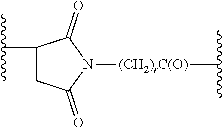

wherein r is an integer ranging from 1 to 10; wherein carbonyl terminus of -A-forming bond with an amino acid unit (W) and the succinimido terminus of -A-forming bond with antibody (Ab).

In a yet another embodiment, the present invention provides antibody-linker-drug conjugates (ADC) wherein mAb is a monoclonal antibody comprising light chain variable region complementarity determining region (CDR) sequences CDR1 (KASQDVSIAVA, SEQ ID NO: 1), CDR2 (SASYRYT, SEQ ID NO: 2) and CDR3 (QQHYITPLT, SEQ ID NO: 3) and heavy chain variable region CDR sequences CDR1 (NYGMN, SEQ ID NO: 4), CDR2 (WINTYTGEPTYTDDFKG, SEQ ID NO: 5) and CDR3 (GGFGSSYWYFDV, SEQ ID NO: 6); enzymatically cleavable linker unit is mc-vc-PABC and drug is drug (D) is a suitable auristatin analogue MMAE.

In a still another embodiment, the present invention provides antibody-linker-drug conjugates (ADC) wherein the ADC structure represented by;

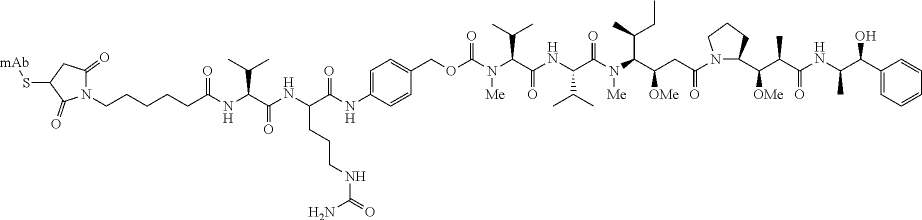

wherein mAb is a monoclonal antibody comprising light chain variable region complementarity determining region (CDR) sequences CDR1 (KASQDVSIAVA, SEQ ID NO: 1), CDR2 (SASYRYT, SEQ ID NO: 2) and CDR3 (QQHYITPLT, SEQ ID NO: 3) and heavy chain variable region CDR sequences CDR1 (NYGMN, SEQ ID NO: 4), CDR2 (WINTYTGEPTYTDDFKG, SEQ ID NO: 5) and CDR3 (GGFGSSYWYFDV, SEQ ID NO: 6).

In a further embodiment, the present invention provides antibody drug conjugate (ADC) wherein mAb is a monoclonal antibody comprising constant domains and a hinge domain of a human IgG1 antibody. In a still further embodiment, the present invention provides antibody drug conjugate (ADC) wherein mAb is a monoclonal antibody comprising constant domains and a hinge domain of a human IgG1 antibody and wherein one or more amino acids in the Fc portion are mutated.

In an embodiment, the present invention provides antibody drug conjugate (ADC) wherein mAb is the monoclonal antibody, Sacituzumab or variants thereof.

In an embodiment, the present invention provides antibody drug conjugate (ADC), wherein said mAb is attached to between 1 and 8 drug moieties, or 3 to 5 drug moieties or in the range of 3.5 to 4.5 drug moieties preferably in the range of 4.2 to 4.4.

In another embodiment, the present invention provides a pharmaceutical composition comprising antibody drug conjugate (ADC) as claimed in any preceding claims and an acceptable carrier.

In still another embodiment, the present invention provides a process for preparing the antibody drug conjugate wherein process comprising a) purification of antibody, sacituzumab b) partial reduction of sacituzumab c) conjugation of reduced sacituzumab with vc-MMAE drug linker.

In a yet another embodiment, the present invention provides antibody drug conjugate (ADC) wherein said conjugate is in a form suitable for parenteral administration such as intravenous (i.v), intramuscular (i.m), subcutaneous (s.c.), intraperitoneal (i.p) or any other suitable route of administration.

In a further embodiment, the present invention provides antibody drug conjugate (ADC) wherein the ADC is used to treat cancer.

In a still further embodiment, the present invention provides antibody drug conjugate (ADC) wherein the ADC is used to treat an infection with a pathogenic organism.

In a yet another embodiment, the present invention provides antibody drug conjugate (ADC) wherein the ADC is used to treat an autoimmune disease.

In a further embodiment, the present invention provides antibody drug conjugate (ADC) wherein the ADC is administered as monotherapy or in combination with one or more therapeutic agents selected from the group consisting of unconjugated antibodies, radiolabelled antibodies, drug-conjugated antibodies, toxin-conjugated antibodies, gene therapy, chemotherapy, therapeutic peptides, oligonucleotides, localized radiation therapy, surgery and interference RNA therapy.

In an embodiment, the present invention provides antibody drug conjugate (ADC) wherein the dose of the ADC is about 0.1 mg/kg to 10 mg/kg.

In another embodiment, the present invention provides antibody drug conjugate (ADC) is sacituzumab vedotin.

### EXAMPLES

### Example 1: Preparation of Sacituzumab-Linker-Drug Conjugate

Sacituzumab was expressed in CHO cells and purified by conventional column chromatography comprising r-Protein A affinity column chromatography. Purity of sacituzumab after affinity column purification was found to be >98.5%, as assessed by HP-SEC. Conjugation of drug to sacituzumab (at >1 mg/mL) was performed on thiol (—SH) group under reducing conditions by molar excess (with respect to protein) of valine-citrulline coupled MMAE drug at temperature between 2° C. and 30° C. at pH between 5.0 and pH 8.5. The linker-drug complex was dissolved in 5-20% acetonitrile before mixing to the protein solution. The linker-drug complex can be also dissolved in other organic solvent like dimethyl acetate or dimethyl sulfoxide. The conjugation reaction was performed for about 60 minutes. Post-conjugation, the antibody drug conjugate (sacituzumab-MMAE conjugate) was buffer exchanged, filtered through a 0.2μ filter and stored under frozen condition at −25±5° C.

TABLE 1
Purity of sacituzumab drug conjugate by HP-SEC analysis

| | HP-SEC analysis | | |
|---|---|---|---|
| Sample | HMW | Principal Peak | LMW |
| Sacituzumab-MMAE conjugate | 2.58% | 94.67% | 2.75% |

The antibody drug conjugate was observed to show about 94% purity on HP-SEC analysis as mentioned in table 1.

The sacituzumab-MMAE conjugate can also be prepared by method as described in Example 2.

### Example 2: Method of Preparing Sacituzumab-MMAE Conjugate

Sacituzumab monoclonal antibody was expressed in Chinese Hamster Ovary (CHO) cells. Purification was carried out using conventional column chromatography steps such as, r-Protein A affinity column chromatography, cation exchange chromatography and mixed-mode column chromatography. After purification, purity of the antibody was determined more than 99% using High-Performance Size Exclusion Chromatography (HP-SEC).

Conjugation of vcMMAE Drug to Sacituzumab:
Purified sacituzumab monoclonal antibody was subjected to chemical conjugation with vc-MMAE. vc-MMAE matrix is also known as vedotin, which was purchased from market. The process steps are described as below;
Thawing of the Critical Intermediate (CI, i.e. Sacituzumab)
The CI was stored in frozen form in cryo-bags. Thawing was performed manually in the same cryo-bags. Upon thawing, the CI (sacituzumab) material was transferred to a suitable container for the next step of reaction.

### Partial Reduction of the CI by TCEP

Sacituzumab was subjected to partial reduction (inter-chain S—S cross-links) in the presence of molar excess amount of tris (carboxyethyl) phosphine [TCEP] for about 300 min. Incubation temperature was maintained at or below 37° C., under stirring condition (200 rpm), for the reaction. At the end of reaction, the protein mixture was passed through a 0.2μ filter and collected in a clean de-pyrogenated glass bottle (2 L) to perform the next reaction step.

### Conjugation of the Drug-Linker to the CI

TCEP-treated sacituzumab was incubated with molar excess amount of the drug-linker, vc-MMAE (valine-citrulline monomethyl auristatin E). Incubation was performed under cold condition for NMT 120 min, with gentle stirring (200 rpm). Conjugation takes place with the formation of covalent thioether bond between the free-SH groups (inter-chains) of partially reduced sacituzumab protein and maleimide group of the drug linker, vcMMAE. At the end of conjugation reaction, the reaction mixture was passed through a 0.2 μm filter and subjected to UF/DF for the removal of unreacted drug-linker molecules.

### Removal of Excess Drug-Linker and Buffer Exchange by UF/DF

Post-filtration, the crude reaction mixture containing sacituzumab vedotin was subjected to UF/DF for the removal of unreacted vcMMAE drug-linker, followed by final buffer exchange. For UF/DF, 30 kDa MWCO membrane filter were used. UF/DF was carried out at a maximum transmembrane pressure (TMP) of 0.50 bar. Constant volume diafiltration was carried out with 20 mM Na-citrate buffer of pH 6.6±0.1; conductivity 4.5±0.5 mS/cm, for maximum 30 dia-volumes. Diafiltration was monitored and controlled for pH and conductivity of the retentate to achieve the target values. After achieving the target pH and conductivity, sacituzumab vedotin was recovered at about 7 mg/mL, and passed through a 0.2μ filter.

### Preparation of Formulated Bulk

The concentrated protein solution was collected in a clean depyrogenated glass bottle. To it, trehalose was added from freshly prepared 30% stock solution (0.2μ filtered) to achieve a final concentration of 7% (w/v). Subsequently, polysorbate 80 was added to a final concentration of 0.02% and the final volume of bulk was adjusted with addition of the drug substance buffer to achieve a protein concentration of 5 mg/mL for sacituzumab vedotin. The formulated bulk of sacituzumab vedotin was passed through a 0.2 μm filter, under bio-safety cabinet, and collected in single-use, sterile, non-pyrogenic polyethylene terephthalate glycol (PETG) bottle with white high-density polyethylene (HDPE) screw closure. The filtered formulated bulk was labeled as the sacituzumab vedotin drug substance.

### Example 3: Purity of Sacituzumab Vedotin by HP-SEC Analysis

Sacituzumab vedotin drug substance samples of example 2 were analyzed by analytical HP-SEC to assess the level of purity and presence of product-related size variants (HMW and LMW species), under native conditions. Samples were injected onto a silica column (7.8 mm×30 cm; 3 μm) column for HPLC analysis. The silica matrix used in this column is coupled with hydrophilic diol-type bonded surface chemistry. HP-SEC analysis was performed in isocratic mode with phosphate buffer of pH 6.8, containing 250 mM salt and a polar solvent (15%), as mobile phase. Separation of protein size variants was conducted at a flow rate of 0.5 mL/min, 25° C. column temperature, and recorded with UV detection, at 214 nm.

Purity of sacituzumab vedotin in the sacituzumab drug substance samples was found to be >97.0%. Samples were found to show <1.5% HMW and <2% of LMW species variants, as illustrated in below table 2 and FIG. 1.

TABLE 2
% purity by HP-SEC

| | | % Principal | |
|---|---|---|---|
| Samples | % HMW | Peak | % LMW |
| Sacituzumab-MMAE conjugate 01 | 1.04 | 97.57 | 1.39 |
| Sacituzumab-MMAE conjugate 02 | 0.86 | 97.81 | 1.33 |
| Sacituzumab-MMAE conjugate 03 | 0.74 | 98.06 | 1.20 |

### Example 4: Drug Antibody Ratio (DAR) of Sacituzumab-MMAE Conjugate

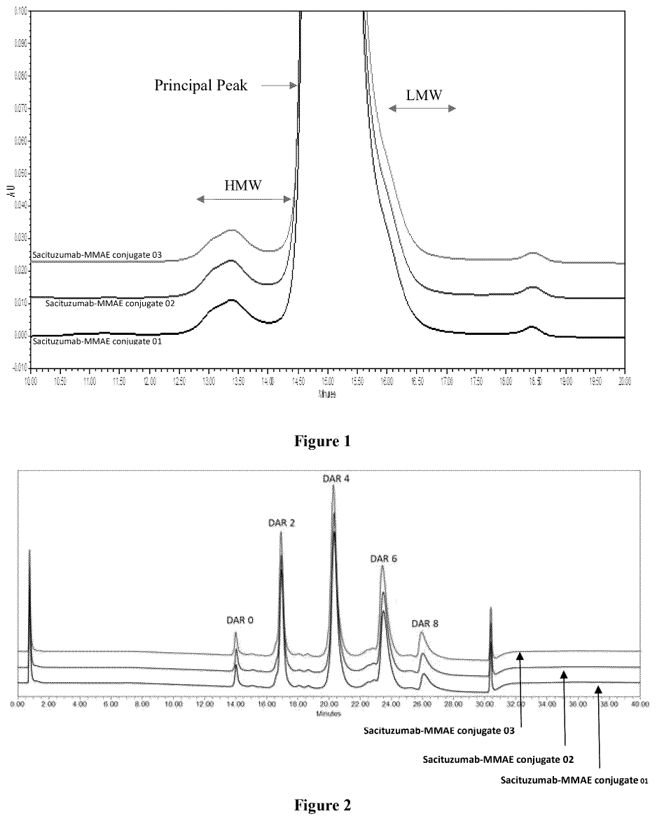

Sacituzumab vedotin drug substance samples of example 3 were analyzed by analytical Hydrophobic Interaction Chromatography (HIC)-HPLC to assess the distribution of drug-linker (DI) on sacituzumab. The peaks are separated by HIC on the basis of relative hydrophobicity of the drug-conjugated-antibody (ADC) carrying 0 to 8 payloads (drug toxin). This technique also allows the determination of average drug-to-antibody ratio (DAR) in sacituzumab vedotin. Experiments were performed by using a butyl (4.6×100 mm; 5 μm) column coupled with HPLC system. Samples containing sacituzumab vedotin drug substance were injected onto the HIC column that was saturated with mobile phase A (50 mM phosphate pH 7.0 and 1.2 M ammonium sulfate buffer containing 5% iso-propyl alcohol), at 25° C. Sacituzumab vedotin carrying varying number of payloads were eluted out of the column with mobile phase B (50 mM phosphate of pH 7.0 containing 20% iso-propyl alcohol), in decreasing salt concentration mode, at 1.0 mL/min, and elution was recorded with UV detection, at 214 nm. FIG. 2 illustrates the appearance of drug-linked antibody variants obtained with three independent batches of sacituzumab vedotin.

All three sacituzumab vedotin drug substance samples were found to show five prominent peaks, which correspond to DAR0, DAR2, DAR4, DAR6 and DAR8, as shown in FIG. 2. Retention time (RT) values of the individual peaks obtained with the batch samples were observed to be nearly identical to each other. Sacituzumab vedotin obtained from all the batches derived were showing an average DAR 4±1, preferably DAR about 4 to 4.5, or DAR about 3.5 to 4.5, preferably 4.2 to 4.4, more preferably DAR about 4.3 on HP-HIC analysis as provided in below table 3 and FIG. 2.

TABLE 3
% Drug antibody ratio (DAR) of sacituzumab-MMAE conjugate

| | | | | | | Average |
|---|---|---|---|---|---|---|
| Samples | % DAR 0 | % DAR 2 | % DAR 4 | % DAR 6 | % DAR 8 | DAR |
| Sacituzumab-MMAE conjugate 01 | 2.96 | 21.02 | 41.92 | 26.97 | 7.12 | 4.29 |
| Sacituzumab-MMAE conjugate 02 | 2.93 | 20.45 | 41.10 | 26.96 | 8.56 | 4.36 |
| Sacituzumab-MMAE conjugate 03 | 2.73 | 19.93 | 40.46 | 27.48 | 9.40 | 4.42 |

### Example 5: Polypeptide Profile of Sacituzumab-Linker-Drug Conjugate Using SDS PAGE Electrophoresis

Preparation of sacituzumab-linker-drug conjugate: Sacituzumab-linker-drug conjugate was prepared according to steps described in Example 1.

### SDS Page Electrophoresis:

Instrument: BIO-RAD electrophoresis System.
Electrophoresis Buffer composition: 0.0247 M Tris, 0.192 M glycine, 0.1% SDS, pH 8.3±0.1.
Sample Preparation: Each sample along with Internal Reference standard was diluted with Milli Q water and 5× sample buffer in such a way that, while loading sample each gel well will be loaded with 10 μg protein.

### Polypeptide Profile of Sacituzumab-Linker-Drug Conjugate (Sacituzumab ADC):

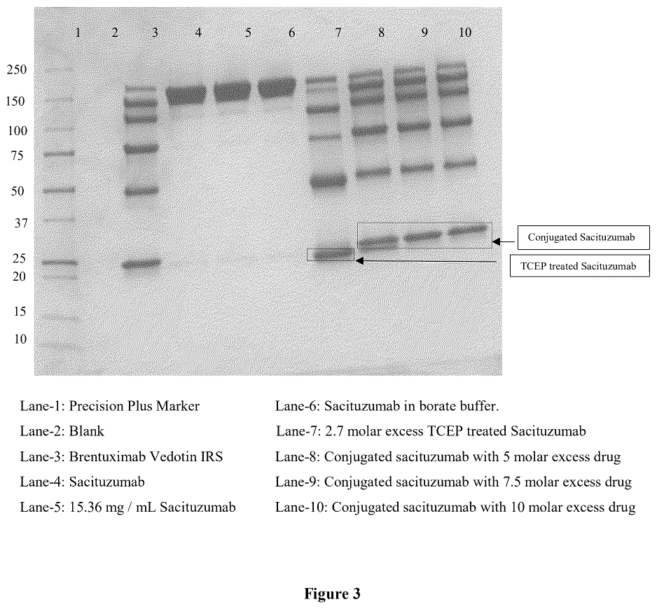

Polypeptide profile of sacituzumab ADC has been demonstrate in FIG. 3. It describes the results of SDS-PAGE analyses of various samples obtained during conjugation reaction of sacituzumab ADC, including the purified sacituzumab in lane no 4, 5 and 6. SDS-PAGE analyses of the samples were performed under non-reducing conditions. Internal reference standard i.e. Brentuximab Vedotin (ADC) was also analyzed under the same conditions, as positive control (Lane No. 3). Post SDS-PAGE analyses, it has been observed that sacituzumab is intact in borate buffer after buffer exchange as single band is observed in lane no. 6. Post partial reduction with 2.7 molar excess TCEP, multiple bands of heterogenous species of sacituzumab are observed a seen in Lane No. 7. The conjugated sample in lane no 8, 9 and 10 signifies the successful conjugation as each band with payload shifts towards upper side i.e. higher molecular weight compared to TCEP treated sample (lane no 7).

### Example 6: In-Vitro Cytotoxicity Study of Sacituzumab-Linker-Drug Conjugate

Example 6A: In-Vitro Cytotoxicity Assay of Sacituzumab-MMAE Conjugate Using BxPC3 Cells

Sacituzumab-MMAE conjugate was prepared and purified as mentioned in example 1. The sacituzumab-MMAE was studied in an in-vitro cytotoxicity assay as mentioned herein below.

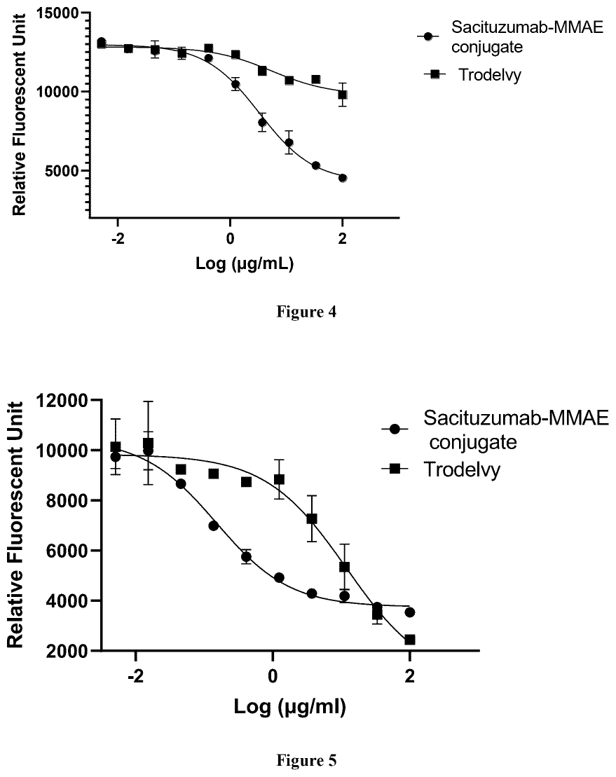

BxPC3 (ATCC CRL-1687, Pancreatic Carcinoma) cells were seeded on a 96 well black clear bottom plate (Costar 3603) and incubated in a CO₂ incubator for adherence. The BxPC3 cell line was chosen as it is known to express the trop-2 antigen. The adherent cells were treated with different concentrations of sacituzumab-MMAE conjugate and Trodelvy®, and incubated in a CO₂ incubator for 2 hours. After incubation the excess or unbound drug conjugates were removed, fresh media added and the plate incubated for 4 days in 5% CO₂ at 37° C. At the end of incubation, alamar blue dye (invitrogen, DAL-1100) was added to the cells and incubated for 5 hours in CO₂ incubator. After incubation viable cells were quantified by measuring the fluorescence signals at an excitation wavelength of 530 nm and an emission wavelength of 590 nm using a SpectraMax M2E fluorescent plate reader. FIG. 4 shows that the BxPC3-cytotoxic activity of the tested sacituzumab-MMAE conjugate is significantly better than Trodelvy®.

### Example 6B: In-Vitro Cytotoxicity Assay of Sacituzumab-MMAE Using NCI-N87 Cells

Sacituzumab-MMAE conjugate was prepared and purified as mentioned in example 1. The sacituzumab-MMAE was studied for in-vitro cytotoxicity assay as mentioned herein below. NCI-N87 (ATCC CRL-5822, Gastric Carcinoma) cells were seeded on a 96 well black clear bottom plate (Costar 3603), and incubated in a CO₂ incubator for adherence. The NCI-N87 cell line was chosen, as it is known to express the trop-2 antigen. The adherent cells were treated with different concentrations of sacituzumab-MMAE and Trodelvy®, and incubated in CO₂ incubator for 2 hours. After incubation the excess or unbound conjugates were removed, fresh media added and incubated for 4 days in 5% CO₂ at 37° C. At the end of incubation, alamar blue dye (invitrogen, DAL-1100) was added to the cells and incubated for 5 hours in CO₂ incubator. After incubation viable cells were quantified by measuring the fluorescence signals at an excitation wavelength of 530 nm and an emission wavelength of 590 nm using a SpectraMax M2E fluorescent plate reader. FIG. 5 shows that the NCI-N87-cytotoxic activity of the tested sacituzumab-MMAE conjugate was significantly better than Trodelvy®.

### Example 6C: In-Vitro Cytotoxicity Assay of Sacituzumab-MMAE Using HCC1806 Cells

Sacituzumab-MMAE conjugate was prepared and purified as mentioned in examples herein above. The sacituzumab-MMAE was studied for in-vitro cytotoxicity assay as mentioned herein below.

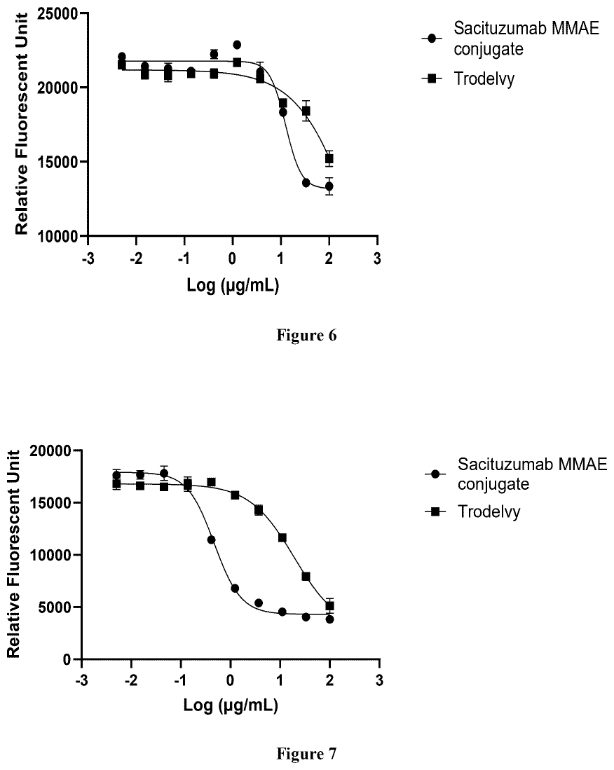

HCC1806 (ATCC CRL-2335, Triple Negative Breast Acantholytic Squamous Cell Carcinoma) cells were seeded on a 96 well black clear bottom plate (Costar 3603), and incubated in a CO₂ incubator for adherence. The HCC1806 cell line was chosen, as it is known to express the trop-2 antigen. The adherent cells were treated with different concentrations of sacituzumab-MMAE and Trodelvy®, and incubated in CO₂ incubator for 2 hours. After incubation the excess or unbound conjugates were removed, fresh media added and incubated for 4 days in 5% CO₂ at 37° C. At the end of incubation, alamar blue dye (invitrogen, DAL-1100) was added to the cells and incubated for 5 hours in CO₂ incubator. After incubation viable cells were quantified by measuring the fluorescence signals at an excitation wavelength of 530 nm and an emission wavelength of 590 nm using a SpectraMax M2E fluorescent plate reader. FIG. 6 shows that the HCC1806-cytotoxic activity of the tested sacituzumab-MMAE conjugate was significantly better than Trodelvy®.

### Example 6D: In-Vitro Cytotoxicity Assay of Sacituzumab-MMAE Using MDA-MB-468 Cells

Sacituzumab-MMAE conjugate was prepared and purified as mentioned in examples herein above. The sacituzumab-MMAE was studied for in-vitro cytotoxicity assay as mentioned herein below.

MDA-MB-468 (ATCC HTB-132, Triple Negative Breast Adenocarcinoma) cells were seeded on a 96 well black clear bottom plate (Costar 3603), and incubated in a CO₂ incubator for adherence. The MDA-MB-468 cell line was chosen, as it is known to express the trop-2 antigen. The adherent cells were treated with different concentrations of sacituzumab-MMAE and Trodelvy®, and incubated in CO₂ incubator for 2 hours. After incubation the excess or unbound conjugates were removed, fresh media added and incubated for 4 days in 5% CO₂ at 37° C. At the end of incubation, alamar blue dye (invitrogen, DAL-1100) was added to the cells and incubated for 5 hours in CO₂ incubator. After incubation viable cells were quantified by measuring the fluorescence signals at an excitation wavelength of 530 nm and an emission wavelength of 590 nm using a SpectraMax M2E fluorescent plate reader. FIG. 7 shows that the MDA-MB-468-cytotoxic activity of the tested sacituzumab-MMAE conjugate was significantly better than Trodelvy®.

### Example 7: In-Vivo Efficacy of Sacituzumab-MMAE Conjugate in Gastric Carcinoma Xenograft Disease Model in SCID Mice

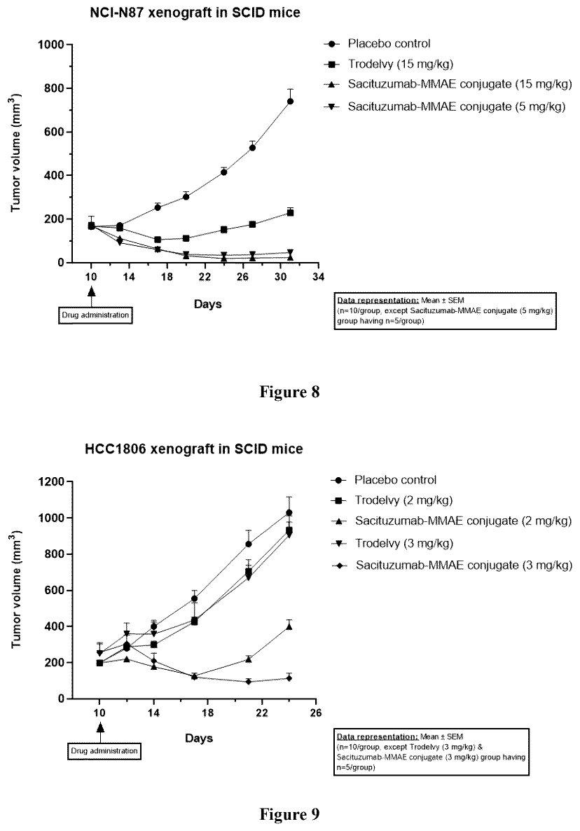

In present study, the therapeutic potential of novel Sacituzumab-MMAE conjugate was evaluated in comparison with the commercially available anti-trop 2 ADC molecule, sacituzumab govitecan-hziy (TRODELVY®). Specifically to induce gastric carcinoma xenograft, immunodeficient SCID mice were injected subcutaneously with NCI-N87 cells (10 million cells/animal/200 μL volume in a 1:1 ratio of cell suspension & matrigel) in the right flank region of animals on day 0. Animals were observed for palpable tumor development & drug administration was done on day 10 when mean tumor volume of animals reached to approximately 150-200 mm³. Sacituzumab-MMAE conjugate and Trodelvy® were compared head to head at a dose level of 15 mg/kg (I.V. route, Single administration) and one additional group of Sacituzumab-MMAE conjugate was tested at a much lower dose of 5 mg/kg (I.V. route, Single administration). FIG. 8 demonstrates the efficacy of Sacituzumab-MMAE conjugate in comparison to Trodelvy® in NCI-N87 gastric carcinoma cell line. Trodelvy® at a dose of 15 mg/kg was able to delay the tumor progression in comparison to placebo control group but not able to regress the tumor substantially. On the other hand, Sacituzumab-MMAE conjugate at both the tested doses was able to regress the tumor burden substantially indicating that even at a one third lower dose, Sacituzumab-MMAE conjugate had greater potential to regress gastric carcinoma tumors carrying TROP2 over-expression as compared to the commercially available drug, Trodelvy®.

### Example 8: In-Vivo Efficacy of Sacituzumab-MMAE Conjugate in BRCA Mutated Triple Negative Breast Cancer Xenograft Disease Model in SCID Mice

In this study, the therapeutic potential of novel Sacituzumab-MMAE conjugate was evaluated in comparison with the commercially available anti-trop2 ADC drug, TRODELVY® in a BRCA mutated triple negative breast cancer (TNBC) disease model. Specifically to induce this TNBC xenograft, immunodeficient SCID mice were injected subcutaneously with HCC1806 cells (10 million cells/animal/200 μL volume, in a 1:1 ratio of cell suspension & matrigel in the right flank region of animals on day 0. Animals were observed for palpable tumor development & drug administration was done on day 10 when mean tumor volume of animals reached to approximately 200-250 mm³. Sacituzumab-MMAE conjugate & Trodelvy® were compared head to head at two different dose levels of 2 mg/kg & 3 mg/kg (I.V. route, single administration). FIG. 9 demonstrates the efficacy of Sacituzumab-MMAE conjugate in comparison to Trodelvy® in BRCA mutated triple negative breast cancer (HCC1806) model. Trodelvy®, at both the tested doses, was only able to slightly delay the tumor progression and not at all able to regress the tumor. On the other hand, Sacituzumab-MMAE conjugate at the dose levels of 2 mg/kg and 3 mg/kg were able to regress the tumor growth up to days 17 and 24 respectively, clearly indicating that Sacituzumab-MMAE conjugate has a greater potential than Trodelvy® to regress & delay tumor progression as a monotherapy in TROP2 over expressing BRCA mutated TNBC tumors.

### REFERENCES INCORPORATED IN CURRENT PATENT APPLICATION

1. Diamantis N, Banerji U. Antibody-drug conjugate—an emerging class of cancer treatment. Br J Cancer 2015; 114:362-367.
2. Bouchard H, Viskov C, Garcia-Echeverria C. Antibody-drug conjugates—a new wave of cancer drugs. Bioorganic Med. Chem Lett 2014; 24:5357-5363.
3. Tolcher A W, Ann Oncol. 2016; 27:2168-2172. 10.1093/annonc/mdw424. [PubMed: 27733376]
4. WHO Drug Information, Vol. 29, No. 2, 2015 Recommended INN: List 63 41 International Non-proprietary Names for Pharmaceutical Substances (INN).
5. Ansel et al, PHARMACEUTICAL DOSAGE FORMS AND DRUG DELIVERY SYSTEMS, 5th Edition (Lea & Febiger 1990), and
6. Gennaro (ed.), REMINGTON'S PHARMACEUTICAL SCIENCES, 18th Edition (Mack Publishing Company 1990), and revised editions thereof.
7. Cardillo et al, Synthetic Lethality Exploitation by an Anti-Trop-2-SN-38 Antibody-Drug Conjugate, IMMU-132, Plus PARP Inhibitors in BRCA1/2-wild-type Triple-Negative Breast Cancer, Clinical Cancer Research; 23 (13); 3405-15. 2017 AACR.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1.-18. (canceled)

19. An antibody-linker-drug conjugate (ADC) wherein antibody is a monoclonal antibody comprising light chain variable region complementarity determining region (CDR) sequences CDR1 (KASQDVSIAVA, SEQ ID NO: 1), CDR2 (SASYRYT, SEQ ID NO: 2) and CDR3 (QQHYITPLT, SEQ ID NO: 3) and heavy chain variable region CDR sequences CDR1 (NYGMN, SEQ ID NO: 4), CDR2 (WINTYTGEPTYTDDFKG, SEQ ID NO: 5) and CDR3 (GGFGSSYWYFDV, SEQ ID NO: 6); linker is enzymatically cleavable linker unit mc-vc-PABC and drug is a suitable auristatin analogue MMAE; wherein said monoclonal antibody (mAb) is attached to between 4.2 and 4.4 drug moieties.

20. The antibody-linker-drug conjugates (ADC) as claimed in claim 19, wherein the ADC is represented by the following structure: 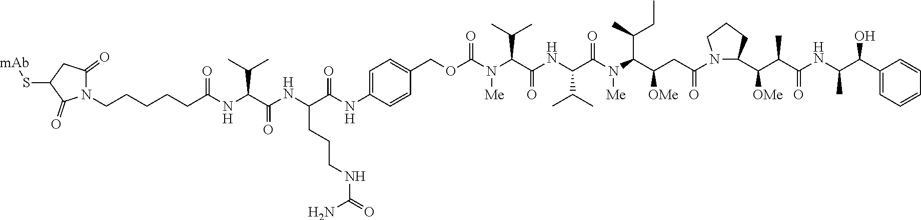
Wherein mAb is a monoclonal antibody comprising light chain variable region complementarity determining region (CDR) sequences CDR1 (KASQDVSIAVA, SEQ ID NO: 1), CDR2 (SASYRYT, SEQ ID NO: 2) and CDR3 (QQHYITPLT, SEQ ID NO: 3) and heavy chain variable region CDR sequences CDR1 (NYGMN, SEQ ID NO: 4), CDR2 (WINTYTGEPTYTDDFKG, SEQ ID NO: 5) and CDR3 (GGFGSSYWYFDV, SEQ ID NO: 6) and wherein said monoclonal antibody (mAb) is attached to between 4.2 and 4.4 drug moieties.

21. The antibody drug conjugate (ADC) as claimed in claim 20, wherein mAb is a monoclonal antibody comprising constant domains and a hinge domain of a human IgG1 antibody.

22. The antibody drug conjugate (ADC) as claimed in claim 21, wherein mAb is a monoclonal antibody comprising constant domains and a hinge domain of a human IgG1 antibody and wherein one or more amino acids in the Fc portion are mutated.

23. The antibody drug conjugate (ADC) as claimed in claim 21 wherein mAb is the monoclonal antibody, Sacituzumab or variants thereof.

24. The antibody drug conjugate (ADC) as claimed in claim 19, wherein said mAb is attached to 4.2 drug moieties, about 4.3 drug moieties or 4.4 drug moieties.

25. The antibody drug conjugate (ADC) as claimed in claim 19, wherein the said mAb is attached to 4.20, 4.21, 4.22, 4.23, 4.24, 4.25, 4.26, 4.27, 4.28, 4.29, 4.30, 4.31, 4.32, 4.33, 4.34, 4.35, 4.36, 4.37, 4.38, 4.39 or 4.40 drug moieties.

26. A pharmaceutical composition comprising antibody drug conjugate (ADC) as claimed in claim 19 and an acceptable carrier.

27. A process for preparing the antibody drug conjugate as claimed in claim 19 wherein process comprising a) purification of antibody, sacituzumab b) partial reduction of sacituzumab c) conjugation of reduced sacituzumab with vc-MMAE drug linker.

28. The antibody drug conjugate (ADC) as claimed in claim 19, wherein said conjugate is in a form suitable for parenteral administration such as intravenous (i.v.), intramuscular (i.m.), subcutaneous (s.c.), intraperitoneal (i.p.) or any other suitable route of administration.

29. The antibody drug conjugate (ADC) as claimed in claim 19 wherein the ADC is used to treat cancer.

30. The antibody drug conjugate (ADC) as claimed in claim 19 wherein the ADC is used to treat an infection with a pathogenic organism.

31. The antibody drug conjugate (ADC) as claimed in claim 19 wherein the ADC is used to treat an autoimmune disease.

32. The antibody drug conjugate (ADC) as claimed in claim 19 wherein the ADC is administered as monotherapy or in combination with one or more therapeutic agents selected from the group consisting of unconjugated antibodies, radiolabelled antibodies, drug-conjugated antibodies, toxin-conjugated antibodies, gene therapy, chemotherapy, therapeutic peptides, oligonucleotides, localized radiation therapy, surgery and interference RNA therapy.

33. The antibody drug conjugate (ADC) as claimed in claim 19 wherein the dose of the ADC is about 0.1 mg/kg to 10 mg/kg.

34. The antibody drug conjugate (ADC) as claimed in claim 19 is sacituzumab vedotin.
